SEQUENCE LISTING (1 sequence)

SEQ ID NO: 1 - protein, 11 aa, Artificial Sequence
  Synthetic peptide
GSNKGAIIGLM

(19) United States
(12) Patent Application Publication
(10) Pub. No.: US 2006/0282906 A1
(43) Pub. Date: Dec. 14, 2006

(54) Non-transgenic rodent model of Alzheimer's disease

(76) Inventors: David F. Cechetto, Ontario (CA); Shawn Whitehead, London (CA); Vladimir Hachinski, London (CA)

Correspondence Address: Licata & Tyrrell P.C., 66 E. Main Street, Marlton, NJ, 08053, US

(21) Appl. No.: 11/149,399

(22) Filed: Jun. 9, 2005

(51) Int. Cl. A01K 67/027

(52) U.S. Cl. 800/12; 800/14

(57) ABSTRACT

The present invention is a non-transgenic rodent exhibiting signs and symptoms associated with Alzheimer's disease. The animal of this invention is produced by acutely injecting β-amyloid (25-35) peptide into the lateral ventricles of a rodent. Animals produced in accordance with this invention can be used in a method of identifying a therapeutic agent for the prevention or treatment of Alzheimer's disease and to study the etiology of this disease.

### BACKGROUND OF THE INVENTION

Alzheimer's disease is associated with various pathologies including neurofibrillary tangles and β-amyloid plaque deposition. β-Amyloid (Aβ) is an insoluble protein which accumulates and forms sticky patches called neuritic plaques. Aβ is derived from the transmembrane amyloid precursor protein (APP). Aβ peptide has both neurotoxic and neurotropic activity. Chemical alterations in both tau and APP proteins need to occur for neurofibrillary tangles and senile neuritic plaques to form.

Various non-transgenic animal models of Alzheimer's disease have been developed. For example, Kowall, et al. ((1992) Neurobiol. Aging 13(5) :537-42) teach injections of Aβ(1-40), and control peptides in rat and monkey cerebral cortex. This reference further teaches injection of Aβ(25-35) in rat cortex. Lesions produced in rat cortex, by solubilized Aβ(1-40) and Aβ(25-35), were generally larger than those produced by control peptides. Tau and ALZ 50 antibodies labeled neurites and diffusely positive perikarya around Aβ(1-40) injections, whereas MAP2 staining was reduced, paralleling the distribution of neuronal loss and gliosis.

Further, both acute and chronic injections of Aβ(25-35) (Olariu, et al. (2001) J. Neural Transm. 108:1065-1079; Yamada and Nabeshima (2000) Pharmacol. Ther. 88:93-113) and Aβ(1-40) or Aβ(1-42) (Frautschy, et al. (1996) Neurobiol. Aging 17(2):311-21; Frautschy, et al. (1998) J. Neurosci. 18:8311-8321; Fukuta, et al. (2001) J. Neural Transm. 108:221-230; Yamada, et al. (1998) Eur. J. Pharmacol. 349:15-22) have been shown to be neurotoxic.

Sigurdsson et al. (1997) Neurobiol. Aging 18(6):591-608) teach bilateral injections of Aβ(25-35) into the amygdala of young male Fischer rats. Aβ induced neuronal tau-2 staining in the right, but not the left amygdala and hippocampus. Aβ also induced reactive astrocytosis and neuronal shrinkage within the right hippocampus and amygdala, respectively. Animals receiving bilateral intra-amygdaloid injections exhibited differences in the numbers of rears with no effect of Aβ in the Morris water maze or in the acquisition and retention of a one-way conditioned avoidance response.

U.S. Pat. No. 6,172,277 discloses a method of inducing amyloid plaque deposition in a rodent by chronic (up to 8 weeks) infusing into the brain of the rodent a solution containing Aβ peptide at a basic pH, wherein the infusion of the peptide results in the formation of amyloid plaques in the brain of said rodent in a greater number than in a control rodent infused with buffer alone or receiving a control peptide.

### SUMMARY OF THE INVENTION

The present invention is a non-transgenic rodent exhibiting signs and symptoms associated with Alzheimer's disease. The rodent of the invention is produced by acute injection of a β-amyloid peptide of SEQ ID NO:1 into the lateral ventricles of said rodent.

The present invention is also a method for identifying a therapeutic agent for the prevention or treatment of Alzheimer's disease. The method involves administering a test agent to a non-transgenic rodent of the present invention, wherein the test agent is administered either before or after injection of the β-amyloid peptide of SEQ ID NO:1 into the lateral ventricles of the rodent, and determining whether the signs or symptoms associated with Alzheimer's disease are prevented or treated in the rodent.

### BRIEF DESCRIPTION OF THE INVENTION

It has now been found that a single cerebroventricular injection of β-amyloid fragment (25-35), referred to herein as Aβ(25-35), elicits increases in pathological and inflammatory correlates of Alzheimer's disease in multiple forebrain sites and decreases performance in the Montoya staircase and Barnes circular platform behavioral test.

Non-transgenic rodent models of the instant invention were generated by acute bilateral injection of 50 nmol of β-amyloid fragment (25-35) (i.e., Gly-Ser-Asn-Lys-Gly-Ala-Ile-Ile-Gly-Leu-Met; SEQ ID NO:1) into the lateral ventricles of rats. Aβ(25-35) injections increased APP-positive staining in hippocampal and neocortical areas. In the cerebral cortex, single cells and diffuse intense clumps ˜20 to 25 μm in diameter were stained for APP. APP signal also increased in the corpus callosum, fornix/anterior hippocampus, and dentate gyrus (Table 1 and Table 2). Increases in Tau-2 signal in the cortex were both cellular and clustered in appearance. Tau-2 signal was also present in the corpus callosum. The CA2 region of the Aβ-treated rat hippocampus showed positive fibrillar type immunostaining for Tau-2 (Table 1).

TABLE 1

| | | | | | Aβ- |
|---|---|---|---|---|---|
| Brain Area | Protein | Sham | Endothelin | Aβ | Endothelin |
| Cortex | GFAP | n.s. | + − | + − | ++ − |
| | OX-6 | n.s. | n.s. | + ↓ | ++ − |
| | APP | n.s. | n.s. | + ↓↓ | +++ − |
| | Tau-2 | n.s. | n.s. | + ↓↓ | + − |
| | TNFα | n.s. | n.s. | + ↓ | + ↓ |
| | NKκB(p65) | n.s. | n.s. | + ↓ | + − |
| | COX-2 | n.s. | n.s. | + ↓↓ | +++ ↓ |
| Corpus | GFAP | n.s. | + − | + ↓↓ | ++ − |
| Callosum | OX-6 | n.s. | n.s. | + ↓↓ | ++ ↓ |
| | APP | n.s. | n.s. | + ↓↓ | ++ − |
| | Tau-2 | n.s. | n.s. | + ↓↓ | + − |
| | TNFα | n.s. | n.s. | + ↓↓ | + − |
| | NKκB(p65) | n.s. | n.s. | + ↓↓ | + ↓ |
| | COX-2 | n.s. | n.s. | + ↓↓ | +++ ↓ |
| Thalamus | OX-6 | n.s. | + − | + − | + − |
| (right) | TNFα | n.s. | + − | + − | + − |
| Hypothalamus | APP | n.s. | n.s. | + ↓ | ++ − |
| | TNFα | n.s. | n.s. | + ↓↓ | ++ ↓ |
| | NKκB (p65) | n.s. | n.s. | + ↓↓ | ++ ↓ |
| Striatum | GFAP | n.s. | ++ − | + − | ++ − |
| (right) | OX-6 | n.s. | ++ ↓↓ | n.s. | ++ ↓ |
| | APP | n.s. | + ↓↓ | n.s. | + ↓ |
| | TNFα | n.s. | + ↓ | n.s. | + ↓ |
| | NKκB(p65) | n.s. | + ↓ | n.s. | + ↓ |
| | COX-2 | n.s. | + ↓↓ | n.s. | ++ ↓ |
| | Cas-3 | n.s. | + ↓ | n.s. | + |
| Hippocampus | GFAP | n.s. | + − | + − | +++ − |
| | OX-6 | n.s. | n.s. | + ↓ | +++ − |
| | APP | n.s. | n.s. | + ↓ | ++ − |
| | Tau-2 | n.s. | n.s. | + ↓ | + − |
| | NKκB(p65) | n.s. | n.s. | + ↓ | + − |
| | COX-2 | n.s. | n.s. | + ↓ | ++ − |

n.s., no positive staining;
+, moderate immunostaining signal;
++, strong immunostaining signal;
+++, strongest immunostaining signal.
Effects of Triflusal are depicted as −, no effect;
↓, moderate reduction,
↓↓, strong reduction.

TABLE 2

| | | | |
|---|---|---|---|
| Sham | Endothelin | Aβ | Aβ/Endo |

APP Accumulation

| | | | | |
|---|---|---|---|---|
| Left Cortex | 5.8 ± 2.4 | 7.4 ± 1.4 | 36.8 ± 7.8^{y} | 77.5 ± 10.6^{y,z} |
| | (7.3 ± 2.3) | (7.3 ± 1.3) | (12.5 ± 2.6)^{x} | 64.5 ± 10.2) |
| Right Cortex | 5.3 ± 1.3 | 13.8 ± 1.9 | 37.8 ± 5.4^{y} | 92.8 ± 9.8^{y,z} |
| | (5.0 ± 2.7) | (13.3 ± 2.1) | (11.0 ± 2.1)^{x} | (85.8 ± 16.1) |

GFAP Intensity

| | | | | |
|---|---|---|---|---|
| Left Hippocampus | 153.1 ± 4.2 | 152.1 ± 4.1 | 167.5 ± 5.2 | 195.5 ± 6.6^{y,z} |
| | (150.8 ± 5.5) | (148.8 ± 4.5) | (166.8 ± 9.1) | (188.9 ± 4.4) |
| Right Hippocampus | 150.7 ± 4.4 | 166.0 ± 3.8 | 166.5 ± 4.2 | 197.3 ± 3.9^{y,z} |
| | (152.5 ± 7.9) | (150.1 ± 3.6)^{x} | (164.2 ± 10.1) | (188.3 ± 8.2) |

OX6 Intensity

| | | | | |
|---|---|---|---|---|
| Left Hippocampus | 101.2 ± 4.2 | 104.2 ± 3.5 | 116.2 ± 6.7 | 154.4 ± 5.2^{y,z} |
| | (97.9 ± 2.0) | (106.8 ± 6.4) | (109.8 ± 6.8)^{a} | (149.0 ± 7.1) |
| Right Hippocampus | 97.8 ± 2.4 | 116.2 ± 2.9 | 119.3 ± 9.1 | 165.2 ± 12.2^{y,z} |
| | (93.4 ± 1.4) | (97.3 ± 4.5)^{x} | (110.8 ± 4.6)^{x} | (149.4 ± 7.4)^{x} |

Data not expressed in brackets are expressed as the mean ± SEM average.
^{x}statistical significance between vehicle and Trifusal treatment;
^{y}statistical significance between sham and surgical treatment;
^{z}statistical significance between antibody or endothelin group and the combined antibody/endothelin group, p < 0.05 (n = 4 for all groups).

Aβ treatment resulted in reactive microglia and astrocytes in the corpus callosum and cingulate gyrus. Activated microglias were also present in the thalamus and cortex (Table 1). TNF-α and NFκB signals were also increased in the cortex and corpus callosum of Aβ-treated rats.

Endothelin-induced ischemia resulted in an extensive increase in immunostaining in the right striatum in the region of the infarct (Table 1 and Table 2). Reactive astrocytes (GFAP) and microglia (OX-6) covered an extensive region surrounding the infarct. OX-6-positive stained microglia also covered a large area of the striatum. Increases in both APP and Tau-2 immunostaining in the region of ischemic damage were also present. Tau-2 and APP staining showed a similar distribution, intensity, shape, and size, and appeared to be both intracellular and extracellular. Increases in NFκB (p65), IL-1β (Table 1), and TNF-α (Table 1) immunostaining showed a punctate appearance in the region of the infarct.

In combined Aβ/endothelin-treated rats, there was a greater intensity of staining of both OX-6-positive microglia and GFAP-positive astrocytes in the hippocampus, particularly in the CA1 region, compared with endothelin or Aβ-treated rats. Average intensity measurements for GFAP in the hippocampus of combined Aβ-endothelin rats (191.9±4.1) was significantly higher than sham (148.2±2.2), endothelin (152.9±11.0), and Aβ (162.9±8.7). Similarly, average numbers of OX-6-stained microglia in the CA1 of the hippocampus of combined Aβ-endothelin rats (36.0±6.5) was significantly higher than sham (1.8±0.7), endothelin (4.8±0.9), and Aβ (13.4±5.1). Other proteins showed increased signal in combined Aβ/endothelin-treated rats (Table 1 and Table 2).

Aβ-treated rat brains showed increased proliferation of microglia surrounding the lateral ventricles and in the corpus callosum and deposits of congophilic Aβ in the wall of the lateral ventricle. Rats receiving Aβ also had lesions in the fornix, anterior hippocampus, and CA1 hippocampus, which stained amyloid positive with Congo Red. The infarct in endothelin-injected rats was surrounded by microglia cells and stained positively for Congo Red.

After Aβ or the combined Aβ/endothelin injections, there was a significant deficit in the total number of pellets eaten on day 8 compared with the last day of training (day −8), which disappeared by day 9. Sham-treated rats did not show a significant deficit on the first day of re-testing in either paw, whereas Aβ-treated rats showed a significant deficit on day 8 for the ipsilateral paw (73±8% of pretreatment). Endothelin-treated rats showed a significant deficit on the first day of re-testing (day 8) for the paw contralateral to the infarct (66±15% of pretreatment). Although the ipsilateral paw of endothelin-treated rats improved performance during the post-treatment phase (143±18% of pretreatment), the combined Aβ/endothelin-treated rats on day 8 showed deficits in both the ipsilateral (79±12% of pretreatment) and contralateral (71±13% of pretreatment) paws and no improved performance over time in the paw ipsilateral to the stroke. Aβ-treated rats showed a significant deficit on day 8 for the ipsilateral paw (73±8% of pretreatment). Although the Montoya staircase test was designed to detect motor deficits (Montoya, et al. (1991) J. Neurosci. Methods. 36:219-228), these results indicate that the Aβ-treated animals had an impairment in their memory for the task and had to undergo a short relearning phase. This memory deficit is consistent with the pathological and inflammatory changes observed in the hippocampus.

Impairments of learning and spatial memory function were examined using the Barnes circular platform test. Intracerebroventricular injections of 50 nmol Aβ(25-35) resulted in an increased latency (time to enter the escape hole) on the memory test trial (day 8) post-surgery compared to the last trial of training (day −1) pre-surgery (P<0.01). Treatment with an inhibitor of NFκB, pyrrolidine dithiocarbamate (PDTC) treatment at the dosage of 100 mg/kg 30 minutes before Aβ(25-35) injections and 7 days post-surgery prevented the Aβ(25-35) induced increases in latency from the trial 15 of test (day 8) to the trial 14 of training (day −1). The test response of the Aβ(25-35) group treated with PDTC was significantly less (P<0.05) than the Aβ(25-35) group with vehicle treatment and not significantly different from the sham groups.

Having demonstrated that a single bilateral injection of Aβ(25-35) into the lateral ventricles exposes more forebrain regions to the toxic effects of this peptide thereby eliciting pathological and inflammatory responses associated with Alzheimer's disease, the present invention is a non-transgenic rodent model for use in studying the etiology of Alzheimer's disease and the identification of therapeutics for the prevention and treatment of Alzheimer's disease. The non-transgenic rodent of the instant invention is produced by acutely injecting a β-amyloid peptide of SEQ ID NO:1 into the lateral ventricles of the rodent. As used herein, an acute injection is intended to mean a single injection or infusion of β-amyloid peptide that occurs for a period of time of less than 24 hours, or more generally less than 1 hour, or more preferably less than 30 minutes. The β-amyloid peptide can be infused or injected as disclosed herein or using any other well-established method for infusing a drug or agent into the brain.

The amount of β-amyloid peptide of SEQ ID NO:1 infused or injected is generally in the range of 25 to 100 nmol and may be dependent upon the size and type of the animal being injected and the level of toxicity desired. While rodents such as mice, rats, guinea pig and hamsters are encompassed within the scope of the instant invention, it is contemplated that other animals may also be produced in accordance with the method of the present invention including, but not limited to, monkeys, bovine, sheep, rabbits, and dogs.

In accordance with the method of the invention, the β-amyloid peptide of SEQ ID NO:1 is injected into one or both lateral ventricles. As is well-known in the art of brain anatomy, the lateral ventricles are two horseshoe-shaped ventricles, one in each cerebral hemisphere, that communicate with the third ventricle via the foramen of Monro. Injecting Aβ(25-35) into the lateral ventricles has now been shown to expose more forebrain regions to the toxic effects of this peptide. As such, the non-transgenic rodents of the instant invention exhibit a plurality of signs and symptoms associated with Alzheimer's disease, which are not found in control or sham-treated animals. As exemplified herein, such signs and symptoms associated with Alzheimer's disease include APP-positive staining in the hippocampal and neocortical areas; increased tau-2 staining in the cortex, corpus callosum, and hippocampus; reactive microglia and astrocytes in the corpus callosum, cingulate gyrus, thalamus, and cortex; increased expression of TNF-α and NFκB in the cortex and corpus callosum; increased proliferation of microglia surrounding the lateral ventricles and in the corpus callosum; deposits of congophilic Aβ in the wall of the lateral ventricle and CA1 hippocampus; lesions in the fornix, anterior hippocampus, and CA1 hippocampus; and memory deficit. These pathological and inflammatory signs and symptoms are characteristic of Alzheimer's disease in humans.

Non-transgenic rodents of the instant invention are useful for the discovery and development of diagnostics and therapeutic agents for the prevention and treatment of Alzheimer's disease and other neurodegenerative diseases. Accordingly, the present invention is also a method for identifying or screening for therapeutic agents which prevent (i.e., inhibit or delay the development or onset of) or treat Alzheimer's disease. The screening method involves administering a test agent to a non-transgenic rodent of the instant invention either prior to or after injecting Aβ(25-35) into the lateral ventricles of the rodent and determining whether the signs or symptoms associated with Alzheimer's disease are prevented, delayed, or treated as compared to a control animal. A control animal can include a non-transgenic rodent of the invention which has not been administered a test agent, or a sham-treated rodent which has or has not been administered the test agent. In cases where the test agent is administered prior to injecting Aβ(25-35), the non-transgenic rodent is monitored for the prevention or delay of onset of signs or symptoms. In cases where the test agent is administered after injecting Aβ(25-35), the non-transgenic rodent may or may not already be exhibiting signs or symptoms associated with Alzheimer's and the rodent is monitored for the delay of onset or treatment of signs or symptoms. Signs or symptoms of Alzheimer's disease can be monitored by examining the rodent for the appearance of abnormal brain histology or appearance of behavioral changes. A behavioral change includes motor or memory impairment and can be determined, for example, by examining the performance of the animal in a rotarod task, Montoya staircase test, Morris water maze, or Barnes circular platform test as exemplified herein.

Test agents which can be screened in accordance with the screening assay provided herein encompass numerous chemical classes, though typically they are organic molecules, preferably small organic compounds having a molecular weight of more than 100 and less than about 2,500 daltons. Libraries of such compounds can contain either collections of pure agents or collections of agent mixtures. Examples of pure agents include, but are not limited to, proteins, antibodies, peptides, peptide aptamers, nucleic acids, oligonucleotides, carbohydrates, lipids, synthetic or semi-synthetic chemicals, and purified natural products. Such libraries are commercially available to the skilled artisan. Examples of agent mixtures include, but are not limited to, extracts of prokaryotic or eukaryotic cells and tissues, as well as fermentation broths and cell or tissue culture supernates.

Animals exhibiting signs and symptoms associated with Alzheimer's disease can now be produced in a short period of time and used as a model to study possible therapies including pharmaceutical intervention, gene targeting techniques, antisense therapies, antibody therapies, etc. Furthermore, such animals can be used to examine situations or environmental hazards which are suspected of accelerating or initiating Alzheimer's diseases and vascular dementia, such as for example, head trauma, or toxic environmental agents, diabetes, hypertension or atherosclerosis. In this case, the animal may be exposed to a particular situation and then observed to determine motor and cognitive (memory and learning) impairment, premature death, etc. as indicators of the capacity of the situation to further provoke and/or enhance Alzheimer's disease.

The animals of the present invention are useful for the more detailed characterization of Alzheimer's disease. For example, the pathogenesis and sequence of molecular events can be elucidated, thereby leading to better treatments for the disease.

The following non-limiting examples are provided to further illustrate the present invention.

### EXAMPLE 1

### Surgery (Day 0)

Male Wistar rats (250 to 300 grams; Charles River, Montreal, Quebec) were divided into 4 groups: intracerebroventricular injections of Aβ(25-35) (BACHEM, Torrance, Calif.); endothelin injections into the right striatum; both Aβ(25-35) and striatal endothelin injections; and sham procedures. The Aβ(25-35) fragment was used to reduce the possibility of rapid coagulation and to allow diffusion of the peptide into the brain.

All rats were anesthetized using 40 mg/kg of pentobarbital (Somnotol; MTC Pharmaceuticals, Cambridge, Ontario) intraperitoneally. Body temperature was maintained at 37° C. Aβ(25-35) (50 nmol in 10 μL of saline) was injected bilaterally into the lateral ventricles. Aβ(25-35) was prepared according to established methods (Sigurdsson, et al. (1997) supra). Aβ(25-35) (50 nmol in 10 μL of saline at a rate of 1 μL/minute) was injected bilaterally into the lateral ventricles via a stainless-steel cannula (23-gauge; anterior/posterior −0.8, medial/lateral ±1.4, and dorsal/ventral −4.0 below dura). Two endothelin injections (6 pmol in 3 μL of saline), 1 mm apart, were made into the right striatum (Sigurdsson, et al. (1997) supra). Sham procedures involved all the surgical steps without injections of Aβ(25-35) or endothelin. After wound suture, all rats received 40 mg/kg of buprenorphine intramuscularly and were allowed to recover from surgery for 7 days.

### EXAMPLE 2

### Staircase Test

For training (n=10 for each group), rats were placed into the Montoya staircase apparatus (Montoya, et al. (1991) supra) at the same time every day for 8 days (days −8 through −1) and were allowed to perform the test for 20 minutes. Rats were food-deprived for 1 day (day −9) before testing, as well as on day −8. On days −7 though −1, rats were fed 10 grams of their normal chow so that they maintained their body weight at ˜85%. Three NOYES LAB ANIMAL PRECISION FOOD PELLETS™ (Research Diets, Inc., New Brunswick, N.J.) were placed in each of the 14 wells (7 on each side). Pellets eaten were recorded from each well. Re-testing trials, which were performed 7 days after the surgical procedures (day 0), were performed for 8 days in the same way as the training trials (days 8 through 15).

### EXAMPLE 3

### Barnes Circular Platform Test

Learning and memory deficits were assessed using the Barnes circular platform. There are twenty holes evenly located along the border of circular platform with a hidden escape box beneath one of the holes. The circular platform is equipped with colored cues on the surrounding wall. A bright light combined with wind from a fan hung on the ceiling above the platform provides the incentive for the rats to find the hidden box and enter the escape hole. During each trial, the rats were initially placed in the center of the platform under an inverted box, then the box was lifted up and the rat is allowed to find the hole for up to 4 minutes. The latency (time to enter the escape hole) was recorded during the trial. There was a training period (trials 1-14) performed at day −7 to day −1 before surgery (day 0). PDTC or vehicle treatment was given 30 minutes before Aβ(25-35) injections and from days 1 to 7 post-surgery. Spatial memory was then tested on day 8 (trial 15) with the escape hole placed in the same location. Spatial learning was examined by re-testing (re-acquisition) from days 9 to 15 (trials 16-29) with the escape box relocated at 1350 from the original location.

### EXAMPLE 4

### Tissue Processing

Twenty-one days after surgery, all animals were euthanized via pentobarbital overdose and perfused transaortically first with saline followed by 4% formaldehyde (pH 7.4). The brains were removed and cryoprotected in 30% sucrose for 36 hours at 4° C. Coronal sections (30 pm) were cut using a cryostat.

### EXAMPLE 5

### Histochemistry and Immunohistochemistry

Thionine and Congo Red staining procedures were performed after sections being mounted on slides (Kiernan JA. (1999) Histological and Histochemical Methods. Theory and Practice. 3rd ed. Oxford: Butterworth Heinemann).

Free-floating sections from rat brains (n=5 for each group) were treated with 0.03% hydrogen peroxide and blocked with 3 mL of horse or rabbit serum (VECTASTAIN® Elite, Vector Laboratories, Burlingame, Calif.). The following primary antibodies were diluted in filtered phosphate-buffered saline: APPa4 mouse monoclonal (Chemicon International Inc, Temecula, Calif.; 1:1000), GFAP mouse monoclonal (Sigma-Aldrich, St Louis, Mo.; 1:1000), OX-6 mouse monoclonal (Serotec, Inc., Raleigh, N.C.; 1:1000), Tau-2 mouse monoclonal (Sigma-Aldrich, St Louis, Mo.; 1:10000), NFκB(p65) goat polyclonal (Santa Cruz Biotechnologies, Santa Cruz, Calif.; 1:1000), IL-1β goat polyclonal (Santa Cruz Biotechnologies, Santa Cruz, Calif.; 1:1000), and TNF-α-M18 goat polyclonal (Santa Cruz Biotechnologies, Santa Cruz, Calif.; 1:1000). APP-, Tau-2-, GFAP-, and OX-6-stained sections were incubated with 3 mL of horse biotinylated anti-mouse secondary antibody and horse serum. NFκB(p65), IL-1β, and TNF-α-stained sections were incubated with 3 mL of biotinylated rabbit anti-goat secondary antibody and rabbit serum. Sections were then incubated in 3 mL of avidin-biotin complex followed by 0.05% diaminobenzidine. Sections were washed, air-dried, cleared in xylene, and cover-slipped. Brains to be compared were processed at the same time using the same solutions to reduce variability in immunostaining caused by separate processing.

### EXAMPLE 6

### Data Analysis

Digital photographs were taken using a light microscope (Leitz Diaplan). An investigator was blinded to the identification of the rat sections while analysis was being performed. All immunohistochemical data that were compared was processed at the same time to reduce any variation caused by differences of intensity. A grading scale was based on both observable size and intensity of staining (n=5 for each group). Also, relative optical density measurements and numbers of stained cells or accumulations were measured in entire brain areas for quantitative analysis and statistically analyzed with ANOVA and Tukey post hoc test with a significance level of P<0.05. Statistical analysis, using the Student t-test, was performed on the staircase test data with a significance level of P<0.05. The staircase data are expressed as the mean±SEM. Statistical analysis for the Barnes circular platform test involved One-way Analysis of Variance (ANOVA) with Tukey's post hoc test were used to examine the change in latency (time to enter the escape hole) from the trial 15 of test to the trial 14 of training in the Barnes circular platform test, and for the reactivity grade measurement in the immunohistochemical studies. Data are expressed as mean±SEM. A value of P<0.05 using a two-tailed distribution was deemed as statistically significant.

What is claimed is:

1. A non-transgenic rodent exhibiting signs and symptoms associated with Alzheimer's disease, wherein said signs or symptoms are induced by acute injection of a β-amyloid peptide of SEQ ID NO:1 into the lateral ventricles of said rodent.

2. A method for producing a non-transgenic rodent exhibiting signs and symptoms associated with Alzheimer's disease comprising acutely injecting a β-amyloid peptide of SEQ ID NO:1 into the lateral ventricles of a rodent thereby inducing signs and symptoms associated with Alzheimer's disease in the rodent.

3. A method of identifying a therapeutic agent for the prevention or treatment of Alzheimer's disease comprising administering a test agent to a non-transgenic rodent of claim 1, wherein the test agent is administered either before or after injection of the β-amyloid peptide of SEQ ID NO:1 into the lateral ventricles of the rodent, and determining whether the signs or symptoms associated with Alzheimer's disease are prevented, delayed or treated in the rodent thereby identifying a therapeutic agent for the prevention or treatment of Alzheimer's disease.
